(19) United States
(12) Patent Application Publication
(10) Pub. No.: US 2016/0174922 A1
(43) Pub. Date: Jun. 23, 2016

(54) LOW-ENERGY X-RAY IMAGE FORMING DEVICE AND METHOD FOR FORMING IMAGE THEREOF

(71) Applicants: JOB CORPORATION, Kanagawa (JP); NAGOYA UNIVERSITY, Aichi (JP)

(72) Inventors: Yoshie KODERA, Aichi (JP); Tsutomu YAMAKAWA, Kanagawa (JP); Shuichiro YAMAMOTO, Kanagawa (JP); Yoshiharu OBATA, Kanagawa (JP)

(21) Appl. No.: 14/908,178

(22) PCT Filed: Jul. 29, 2014
(86) PCT No.: PCT/JP2014/069906

§ 371 Date: Jan. 28, 2016

(30) Foreign Application Priority Data
Jul. 29, 2013 (JP) 2013-157073

(51) Int. Cl. A61B 6/00

(52) U.S. Cl. CPC A61B 6/502; A61B 6/5258; A61B 6/5205; A61B 6/482

(57) ABSTRACT

A low-energy X-ray image formation apparatus includes an X-ray generator generating X-rays having an energy spectrum showing an energy range continuously ranging from 18-30 keV (or −37 keV), the energy range being higher in energy from an effective energy of an energy range ranging 10 to 23 keV. A detector detects the X-rays transmitted through a soft tissue of a subject or a tissue of a substance. The tissue of the substance corresponds in a contrast-to-noise ratio (CNR) to the soft tissue of the object. A console acquires an image of the soft tissue of the object or of the substance, based on a detection signal from the detector. The soft tissue and the substance are defined as a soft tissue and a substance presenting a CNR of 3.8 or more when the X-rays are radiated in a condition where an X-ray tube voltage is set at 20 kV.

### TECHNICAL FIELD

The present invention relates to a low-energy X-ray image formation apparatus and a method for forming the image, in which soft tissue (soft-part tissue) of an object to be imaged or a substance having a composition of which characteristics pertaining to X-rays correspond to those of the soft tissue is imaged with X-rays. In particular, the present invention relates to a low-energy X-ray image formation apparatus and a method for forming the image, in which X-rays having an energy range that is optimized based on the radiolucency characteristics of the soft tissue are used.

### BACKGROUND ART

In 2011, there were 357,305 cases (male in 213,290 cases and female in 144,115 cases) of death caused by cancer. In particular, cancer that occurs in the breast, primarily that of women, ranks first place (as of 2008) in terms of site-specific prevalence among women in Japan, as well as fifth place (as of 2011) in terms of the number of deaths caused by cancer. The number of such cases is increasing every year. In addition, the risk of developing breast cancer is higher in Europe and the United States, than in Japan. Therefore, breast cancer is an even greater concern in these regions. Should breast cancer be detected at an early stage, prognosis is relatively favorable. Therefore, early detection is an important issue. Consequently, there is a strong call for the necessity of screenings. However, the screening rate in Japan is low, at 12.1% (as of 2010), compared to Europe and the United States. Medical examinations involving the use of mammography, which is considered effective for detection, accounts for merely 10.1% (as of 2010).

Breast cancer screening is mainly conducted through palpation and mammography. However, ultrasonic apparatuses are also effective. Furthermore, magnetic resonance imaging (MRI), computed tomography (CT), biopsy, and the like are used as methods for making a more detailed diagnosis. Among these methods, X-ray mammography is considered to be the easiest, as well as the most effective for early detection.

As described hereafter, intensifying screen film-type X-ray mammography that features high sharpness and high contrast is used to enable detection of tiny calcium depositions and low-contrast tumors. However, in X-ray mammography of recent years, a digital imaging type in which computed radiography (CR) and a flat panel detector (FPD) are used has become mainstream, as a result of advancements in digital technology.

Even in digital X-ray mammography, a resolution of 100 μm or less is required so as to enable detection of calcification (micro calcification), in a manner similar to conventional mammography. At the same time, imaging of slight differences in X-ray absorption with favorable contrast is also required so as to enable detection of mass.

Regarding improvement in resolution related to the former requirement, a method in which detection pixel size is reduced is used. Regarding the latter requirement, because of noise characteristics and limitations to detection sensitivity of a detector, as well as the fact that the breast is mainly composed of soft tissue, modification is made to keep the energy generated from an X-ray generator within a range of 10 to 20 keV, as described in PTL 1. This modification involves the use of molybdenum (Mo) having characteristic X-rays at 17.5 keV and 19.6 keV as a target (anode) material of an X-ray tube. In addition, a molybdenum filter is used. The molybdenum filter suppresses low-energy components in the vicinity of 10 keV that have a significant effect on skin exposure, as well as components at 20 keV and higher that cause reduced contrast. Through use of the effect of suppressing X-rays of energy components at 20 keV and higher as a result of the spectrum of the molybdenum filter having a K-absorption edge at 20 keV, and the effect of suppressing X-rays of low-energy components that is dependent on filter thickness, an X-ray spectrum of a desired energy range (that is, an energy range of 10 to 20 keV) is obtained.

In addition, when the breast is large, a favorable image cannot be obtained unless higher energy is used. Therefore, when the breast is large, rhodium (Rh) that has a slightly higher K-absorption edge at 23.2 keV is used as the filter. In any case, current X-ray mammography can be considered an imaging technique that is weighted by the characteristics at 17.5 keV and 19.6 keV that are the characteristic X-rays of molybdenum.

Meanwhile, in the field of non-destructive inspection, substances that are relatively light in weight, as well as small fish, small animals such as insects, and the like, are imaged by X-rays. In this case as well, X-rays at about 10 keV to 25 keV are similarly used. However, the situation is such that sufficient image quality cannot be obtained unless X-ray tube current is increased or a certain amount of imaging time is secured. In addition, in food product inspection, there is a need for the inclusion of hair in food products to be inspected through X-ray inspection. However, there is still no technique for performing X-ray inspection because of difficulty in achieving image resolution and contrast resolution in a detector that are necessary for visualizing hair (having a thickness of about 70 to 100 μm) and obtaining a number of photons that enable sufficient delineation in-line inspection systems.

In recent years, mammography involving the use of a photon-counting-type detector in which an Si detector is used has been commercialized (Philips Medical Systems; currently distributed in Japan by Canon Inc.). However, because the Si detector has a low specific gravity, significant overhaul is difficult from the perspective of reducing exposure dose from the current X-ray generator conditions. In addition, because the specific gravity of the Si detector is low, when X-ray energy is increased from the current energy band, Compton scattering increases within the Si detector. Even when energy band-specific imaging is performed, a band image corresponding to the energy characteristics is difficult to obtain. Reduced detection sensitivity also becomes an issue.

In addition, in the area of dental X-rays, a panoramic apparatus that uses a CdTe semiconductor detector has been commercialized. The specific gravity of the sensor material is higher than that of Si. Furthermore, in terms of reduced exposure dose, energy of the X-rays to be used may be increased. However, manufacturing a detector that has a pixel size of 100 μm or lower is currently difficult to put into practice, as a result of problems regarding the amount of circuit implementation, increase in the amount of charge sharing, and problems regarding power consumption.

Therefore, in breast imaging, as well as in non-destructive inspection, it can be said that there is no technique for visualizing a target that corresponds to soft tissue and obtaining a fine image resolution of 100 μm or lower, while reducing the amount of X-rays from the current amount.

### CITATION LIST

### Patent Literature

[PTL 1] JP-A-2009-154254

### SUMMARY OF INVENTION

### Technical Problem

In the conventional intensifying screen film type, the three elements of image quality, that is, contrast, sharpness, and noise characteristics can be independently considered. The mammary gland and fat, which are the main tissues of the breast, respectively have X-ray attenuation coefficients of 0.8 cm⁻¹ and 0.45 cm⁻¹ for energy of 20 keV. Conversely, masses and minute calcifications, which make up the main composition of breast cancer, respectively have X-ray attenuation coefficients of 0.85 cm⁻¹ and 1.45 cm⁻¹ for the same energy. Calcification has high contrast in relation to mammary gland tissue, whereas mass has slight contrast. Therefore, to keep this slight contrast at a high level, the energy of the X-rays to be used is reduced, and a high-contrast film is used. Specifically, in many instances, when the breast of the subject has an average thickness (about 4 cm), the X-rays are such that the tube voltage is set to 28 kV. Molybdenum (Mo) is used as the target material. Mo is used as the filter material. Meanwhile, when the breast is thick (about 7 cm), the tube voltage is set to 32 kV. Mo is used as the target material. Rhodium (Rh) is as the filter material.

Here, the following two points pose a problem. First, because the X-ray energy is extremely low, penetrating power is low. Based on calculation the inventors have conducted by Monte Carlo simulation, when X-rays were radiated on a PMMA phantom having a thickness of 4 cm, under conditions in which the tube voltage is 28 kV and the target/filter materials are Mo, when the dose on the phantom surface is 1, the X-rays that pass through the phantom are only 0.0513. When X-rays were radiated on a PMMA phantom having a thickness of 7 cm, under conditions in which the tube voltage is 32 kV, the target material is Mo, and the filter material is Rh, the transmitted X-rays are only 0.0136. In other words, the remaining 99.5% in the former and the remaining 98.6% in the latter were absorbed within the body (exposure), and did not contribute at all to the image. This indicates that most of the X-ray radiation energy is patient exposure, and exposure on the surface of the breast on the side through which the X-rays enter is particularly extremely high.

Furthermore, the magnitude of quantum mottle, which is typical noise in imaging systems, is determined by the average number n of X-ray quanta that is absorbed by the detector, and is 1/v′n. Therefore, noise in an image increases as the number of X-ray quanta absorbed by the detector decreases. Consequently, noise under such imaging conditions becomes extremely high. The contrast effect achieved through energy reduction is compromised. Signal-to-noise ratio (SNR) or contrast-to-noise ratio (CNR) becomes significantly low.

Therefore, to obtain an image that sufficiently enables diagnosis, the X-ray tube current is required to be increased or the X-ray radiation time is required to be increased. When such measures are taken, the X-ray exposure dose to the breast increases. That is, a trade-off relationship is present between a fine image and X-ray exposure dose.

The concern regarding X-ray exposure during breast examination on women is based on such reasons.

Noise characteristics of current detector systems, described above, will be described. Whether a direct-conversion type or an indirect-conversion type, current digital-type detectors mostly use a method in which output from the detector is obtained by the amount of X-rays being integrated over a certain amount of time. In such integrating-type signal detection methods, electrical noise generated when the output from the detector is converted to an electrical signal is also integrated. The electrical noise is typically generated independent of the incident amount of X-rays. Therefore, when the amount of signals (the number of generated X-rays or energy) is small, the weight of the electrical noise component increases. X-ray transmission information tends to become buried in noise and unable to be seen. This tendency is conspicuous in mammography, because the X-ray energy that is used is particularly low.

The present invention has been achieved in light of the issues that occur when soft tissue is imaged with X-rays, that can be seen in the above-described conventional X-ray mammography and the like. Specifically, an object of the present invention is: i) to significantly improve the SN ratio attributed to electrical noise, compared to imaging apparatuses in which conventional integrating-type X-ray detectors are mounted; and ii) to improve circumstances faced by conventional apparatuses in which, contrast is required to be ensured through reduction of X-ray energy, regardless of high X-ray exposure dose to the patient, because the ability to differentiate contrast at high dose regions and low dose regions is insufficient due to the narrow dynamic length of the circuit, or to suppress changes in image quality resulting from changes in the amount of X-rays contributing to imaging that is dependent on the size of the breast.

In addition, in the case of X-ray mammography, an object is to optimize imaging of calcification that has a relatively significantly high X-ray absorption coefficient and mass that has a low X-ray absorption coefficient, which is a characteristic feature of X-ray mammography.

### Solution to Problem

To solve the above-described issues, a low-energy X-ray image formation apparatus of the present invention includes, as a basic configuration, an X-ray generator, a detector, and an image forming means. The X-ray generator generates X-rays having an energy spectrum that is continuously distributed over an energy range that is higher than the effective energy of an energy range from 10 to 23 keV, and that is an energy range having a lower-limit energy value of 18 keV and is from this lower-limit energy value to an upper-limit energy value of 30 keV to 37 keV. The detector detects the X-rays that have been generated by the X-ray generator and have passed through soft tissue to be imaged or a substance having a composition corresponding to the soft tissue from the perspective of contrast-to-noise ratio (CNR). The image forming means forms an image of the soft tissue to be imaged or the substance based on detection signals of the X-rays outputted from the detector. The soft tissue or the substance is defined such as to have the contrast-to-noise ratio (CNR)=3.8 or more when irradiated with the X-rays at an X-ray tube voltage=20 kV, and to be a composition having a similar CNR.

In addition, an image formation method using low-energy X-rays that exhibit functions equivalent to those of the above-described image formation apparatus is also provided.

### Effects of the Invention

In the image formation apparatus and the image formation method of the present invention, problems faced by conventional integrating-type X-ray detectors can be improved. That is, for example, the SN ratio is poor as a result of electrical noise. In addition, contrast is required to be ensured through reduction of X-ray energy, regardless of high X-ray exposure dose to the patient, because the ability to differentiate contrast at high dose regions and low dose regions is insufficient due to the narrow dynamic length of the circuit.

### BRIEF DESCRIPTION OF DRAWINGS

In the accompanying drawings,

FIG. 1 is a diagram for explaining an overview of a configuration of an X-ray mammography apparatus serving as a low-energy X-ray image formation apparatus according to a first embodiment of the present invention;

FIG. 2 is a graph of an example of an energy spectrum of raw X-rays emitted from an anode of an X-ray tube;

FIG. 3 is a graph of an example of an energy spectrum of X-rays emitted from an X-ray tube, after passing through an aluminum filter;

FIG. 4 is a graph for explaining the difference in energy spectrum between X-rays of the present invention and X-rays used in conventional mammography;

FIG. 5 is another graph (including characteristic X-rays) of an energy spectrum of X-rays emitted from an X-ray tube, after passing through an aluminum filter;

FIG. 6 is a diagram for schematically explaining a front view of the apparatus in FIG. 1;

FIG. 7 is a planar view of an overview of an X-ray detector with a partial cutaway view;

FIG. 8 is a perspective view and a cross-sectional view of an overview of a detection module;

FIG. 9 is a block diagram of a data collection circuit individually connected to semiconductor cells forming each pixel;

FIG. 10 is a diagram for explaining a relationship between electrical pulses generated in response to incidence of X-ray photons and a threshold for differentiating the strengths thereof;

FIG. 11 is a diagram for explaining a plurality of energy ranges (BIN) and collection and reconfiguration of X-ray photons for each energy range;

FIG. 12 is a block diagram of an electrical configuration including a console;

FIG. 13 is a diagram for explaining an overview of a configuration of an X-ray foreign matter detection apparatus serving as a low-energy X-ray image formation apparatus according to a second embodiment of the present invention; and

FIG. 14 is a planar view of an overview of an X-ray detector used according to the second embodiment with a partial cutaway view.

### DESCRIPTION OF EMBODIMENTS

Embodiments of a low-energy X-ray image formation apparatus and a method for forming the image of the present invention will hereinafter be described with reference to the accompanying drawings.

In the low-energy X-ray image formation apparatus of the present invention, an object to be imaged is a soft tissue (soft-part tissue) portion of a human body or the like, or a substance that is composed of soft tissue.

Here, the apparatus is given the name “low-energy X-ray image formation apparatus” in the sense that “low-energy” indicates the use of lower X-ray energy, within the energy range of X-rays used in typical X-ray medical diagnostic equipment, excluding conventional mammography. In addition, “formation” in “image formation” is used in the sense that, beyond the concept of imaging an X-ray image, the generation of an image through various processes being performed on the signals of X-rays that have passed through an object and have been received by a detector is included.

Meanwhile, the soft tissue to which the present invention applies refers to soft tissue that has a contrast-to-noise ratio (CNR)=3.8 or higher when irradiated with X-rays at an X-ray tube voltage=20 kV, and a substance that has a similar contrast-to-noise ratio. In the medical field, soft tissue (soft-part tissue) is a term relative to hard tissue, and is defined as a collective term for connective tissue excluding bone tissue. In the present invention, the soft tissue is defined from the perspective of tube voltage and CNR, such as to include this general concept from the medical field. Therefore, the soft tissue referred to in the present invention includes, of course, the human breast, as well as objects to undergo non-destructive inspection such as food products (such as green peppers and other vegetables).

Therefore, the low-energy X-ray image formation apparatus of the present invention is also referred to as an X-ray mammography apparatus or a breast X-ray imaging apparatus when implemented for imaging the human breast. In addition, the low-energy X-ray image formation apparatus has been receiving attention in recent years also as an X-ray foreign matter detection apparatus that serves as a non-destructive inspection apparatus for detecting foreign matter, such as hair, inside food products. In the present application, as the low-energy X-ray image formation apparatus of the present invention, an X-ray mammography apparatus will be described according to a first embodiment and an X-ray foreign matter detection apparatus will be described according to a second embodiment.

### First Embodiment

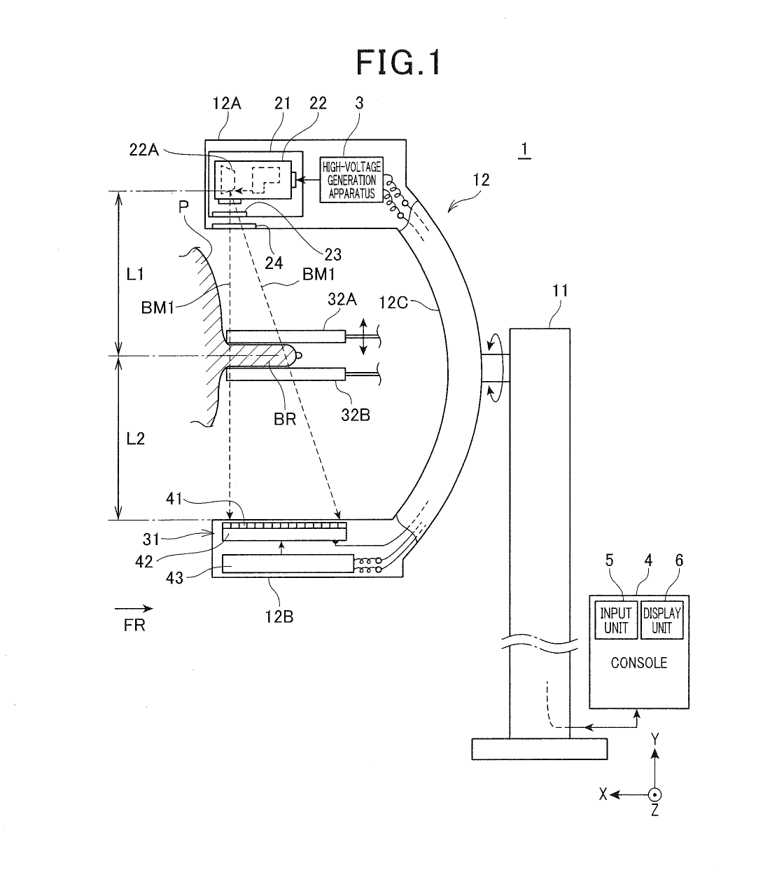

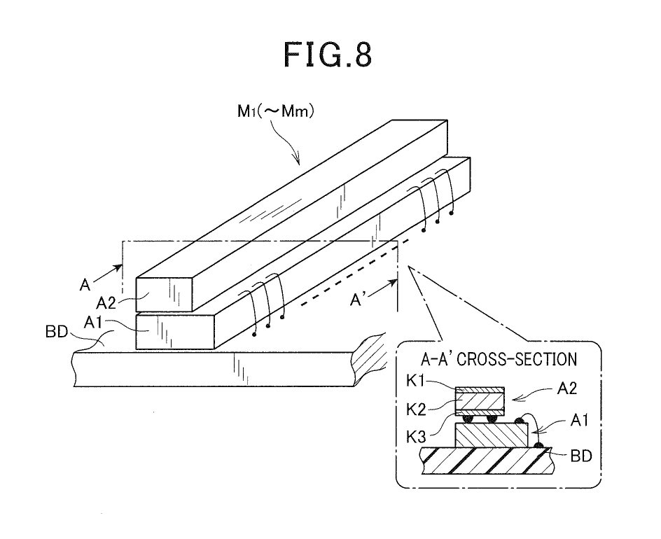

An embodiment of an X-ray mammography apparatus related to the low-energy X-ray image formation apparatus of the present invention will be described with reference to FIG. 1 to FIG. 12.

The X-ray mammography apparatus images the breast of a test subject. In this example, the X-ray mammography apparatus performs X-ray detection by a technique referred to as photon counting. The X-ray mammography apparatus processes the detection value based on a tomosynthesis method and obtains a tomographic image of the breast. Of course, the process for obtaining the image may be that in which a transmission image referred to as a scanogram is obtained. Alternatively, the process may be that in which a computed tomography (CT) image is obtained.

As shown in FIG. 1, an X-ray mammography apparatus 1 according to the present embodiment includes a gantry 11 and an arm portion 12. The gantry 11 stands erect. The arm portion 12 is rotatably held by the gantry 11 such as to be oriented in the lateral direction of the gantry 11. For convenience of description, an orthogonal coordinate system in which the long direction of the gantry 11 is a Y-axis direction is set as shown in FIG. 1.

The arm portion 12 has a substantially C-shaped side surface shape. The arm portion 12 is provided with two upper and lower beam portions 12A and 12B that extend in the lateral direction. The arm portion 12 is also provided with a link portion 12C that connects respective one end portions of the beam portions 12A and 12B in a vertical direction (Y-axis direction). Of these portions, one beam portion 12A is provided with an X-ray generator 21 that generates X-rays. The other beam portion 12B is provided with an X-ray detection apparatus 31 that performs detection by a photon counting method based on the X-rays. In addition, the present apparatus 1 is provided with compression plates 32A and 32B that compress a breast BR of a test subject P into a plate shape. The compression plates 32A and 32B are provided such that the positions thereof in the height direction (that is, the Y-axis direction) is adjustable. The compression plates 32A and 32B are composed of a material having radiolucency.

In addition, the X-ray mammography apparatus 1 also includes a high-voltage generation apparatus 3 and a console 4. The high-voltage generation apparatus 3 supplies an X-ray tube, described hereafter, with a high voltage for driving. The console 4 is used for control and image processing. The high-voltage generation apparatus 3 is disposed inside the above-described beam portion 12A. The console 4 is provided separately from the gantry 11.

The console 4 includes an input unit 5 and a display unit 6 that are used as interfaces by an operator. The console 4 controls the driving units (not shown) of the gantry 11, the arm portion 12, the X-ray detector 31, and the compression plates 32A and 32B. The console 4 also electrically controls the driving of electrical elements within the gantry 11 and the high-voltage generation apparatus 3. Therefore, the console 4 is communicably connected to required components in the gantry 11.

Of these components, the X-ray generator 21 includes an X-ray tube 22 and a filter 23. The filter 23 is successively placed on the X-ray radiation side of the X-ray tube 22. The filter 23 is a filter in which an aluminum (Al) material is formed into a plate shape having a desired thickness. The filter 23 is referred to, hereafter, as an aluminum filter.

The X-ray tube 22 is supplied with the high voltage from the high-voltage generation apparatus 3 that generates the high voltage by inverter control. In the X-ray tube 22, tungsten (W) is used as an anode material 22A thereof.

For example, the above-described X-ray tube 22 emits pulsed X-rays. The X-rays are radiated as pulse-like X-ray beams or a continuous X-ray beam that have been collimated towards the breast BR of the test subject P by the aluminum filter 23 and a collimator (or a slit) 24 (see dotted line BM1 in FIG. 1).

As shown in FIG. 1, the collimator 24 collimates the X-rays such that, of the profile of the X-ray beam BM1, the beam profile on the sternum side of the test subject P is substantially vertical and the profile of the X-ray beam BM1 on the side opposite the sternum side spreads in a fan shape. A reason for this is to enable imaging to be performed as exactly and as closely as possible to the edge of the sternum side of the breast BR, and to prevent excessive X-ray exposure in the region on the sternum side.

In addition, as shown in FIG. 1, the following values are set: focal point-to-subject distance L1=0.5 m; subject-to-detector distance L2=0.5 m; and focal point size of X-ray tube 22=0.056 mm or less. As a result, the magnification factor=2 times, and a phase contrast effect is achieved. Refer to the following literature regarding phase contrast:

### [Non-Patent Literature] Image Quality Characteristics of Phase Contrast Mammography

Investigation of physical image characteristics and phenomenon of edge enhancement by phase contrast using equipment typical for mammography,

Asumi Yamazaki, Katsuhiro Ichikawa, Yoshie Kodera, Medical Physics, 35(11), 5135-5150, 2008.

Here, the voltage to be applied to the X-ray tube 22 is, for example, 30 kV. However, in the present invention, the voltage is set to a value ranging from 30 to 37 kV.

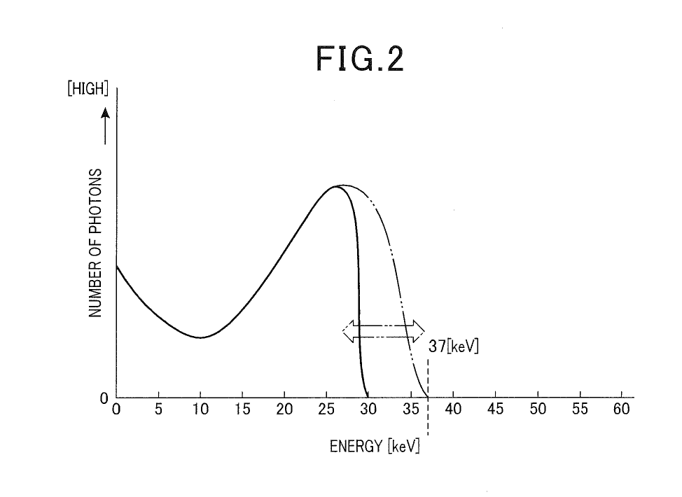

When the tube voltage is 30 to 37 kV, schematically, the energy of the X-rays generated by the X-ray tube 22 itself (that is, the X-rays before passing through the filter 23) has a spectrum such as that shown in FIG. 2. In FIG. 2, a curved line when the tube voltage=30 kV is indicated by a solid line. A curved line when the tube voltage=37 kV is indicated by a virtual line. In this spectrum distribution, energy [keV] is taken on the horizontal axis, and the X-ray photon count is taken on the vertical axis. According to the present embodiment, X-ray detection is performed by the photon counting method. Therefore, the amounts on the vertical axis in the distribution are assigned to photon count (the number of photons).

In the example indicated by the solid line in FIG. 2, the tube voltage is set to 30 kV. Therefore, the upper limit value of energy is 30 keV. The spectrum peak is found midway, near 25 keV. The distribution extends to energy bands lower than 25 keV. That is, a distribution is formed that is continuously broad from energy on the low-band side that is substantially near zero to 30 keV, and has a peak near 25 keV. When the tube voltage is increased or decreased, the intensity and energy of the generated X-rays also increase or decrease by the same extent. That is, depending on the increase and decrease in tube voltage, the height (corresponding to the photon count) and the width (energy value) of the energy spectrum also increases (widens).

This distribution of the energy spectrum, as is, is not suitable for X-ray mammography.

Therefore, the distribution of the energy spectrum of raw X-rays emitted from the X-ray tube 22 is corrected by the aluminum filter 23. That is, the aluminum filter 23 cuts or suppresses the energy spectrum on the low-band side, that is, energy components at about 18 keV and below in this example. The plate thickness of the aluminum filter 23 is selected such as to enable cutting or suppressing of such energy components.

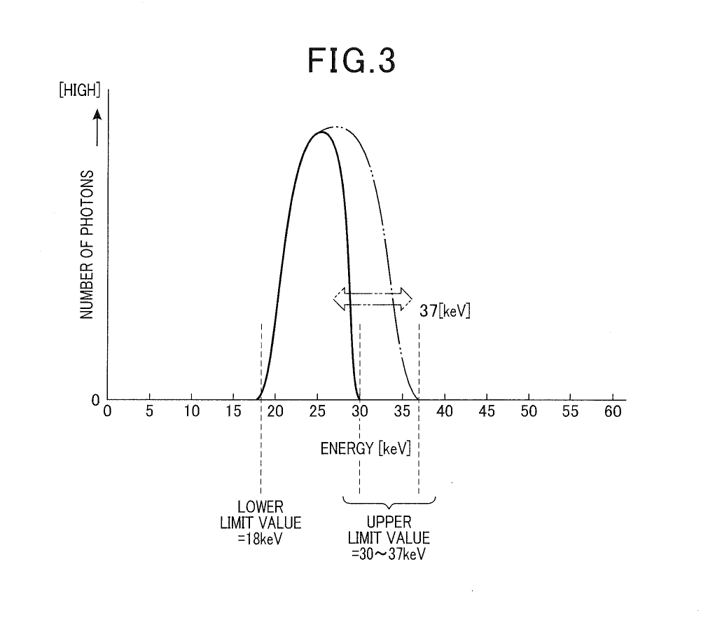

As a result, by passing through the aluminum filter 23, the X-rays emitted from the X-ray tube 22 have an energy spectrum such as that shown in FIG. 3. In FIG. 3, the spectrum distribution on the low-band side is cut by both filters 23. The high-band side is suppressed by the tube voltage 30 kV. Of course, when the tube voltage is set to 37 kV, the energy spectrum widens to 37 keV. According to the present embodiment, as described above, the tube voltage can be arbitrarily set from 30 to 37 keV such as to be selected based on the intentions of the operator. Therefore, as shown in FIG. 3, the X-rays that are radiated outside from the X-ray generator 21 through the collimator 24 have a continuous energy spectrum over a narrow energy range of “lower limit value=18 keV to upper limit value=30 key” to “lower limit value=18 keV to upper limit value=37 key”. Although the spectrum peak is near 25 keV, the peak shifts slightly towards the higher side depending on the value, from 30 to 37 keV, to which the tube voltage is set.

The narrow energy range (“lower limit value=18 keV to upper limit value=30 keV” to “lower limit value=18 keV to upper limit value=37 key”) is set to enable optimal imaging of soft tissue as defined in the present invention, from the perspective of achieving both noise resistance and high contrast. That is, the energy range is such that a contrast-to-noise ratio (CNR)=3.8 or higher can be achieved when the soft tissue is irradiated with X-rays at an X-ray tube voltage=20 kV.

The value CNR=3.8 or higher has been set by the inventors of the present invention and the like as a value that also includes, in addition to medical soft tissue, substances other than a living being that correspond to the soft tissue (referred to, hereafter, as a substance corresponding to soft tissue), from the perspective of foreign matter detection, taking into consideration the X-ray absorption coefficients and densities of normal mammary gland tissue, masses, human hair present as foreign matter, and the like, with reference to resources such as “M. Ishida, et al., ‘Digital Image Processing: Effect on Detectability of Simulated Low-Contrast Radiographic Patterns’, Radiology 1984; 150: 569 to 575”. An example of a substance corresponding to soft tissue is human hair. Hair is given as a representative example of an object that is thin and small, while having a rather high X-ray absorption coefficient.

At the same time, in determining a narrow energy range, a condition has also been considered in that the energy range is higher than the effective energy of the energy range of 10 to 23 keV, from the perspective of mitigating issues faced by conventional mammography.

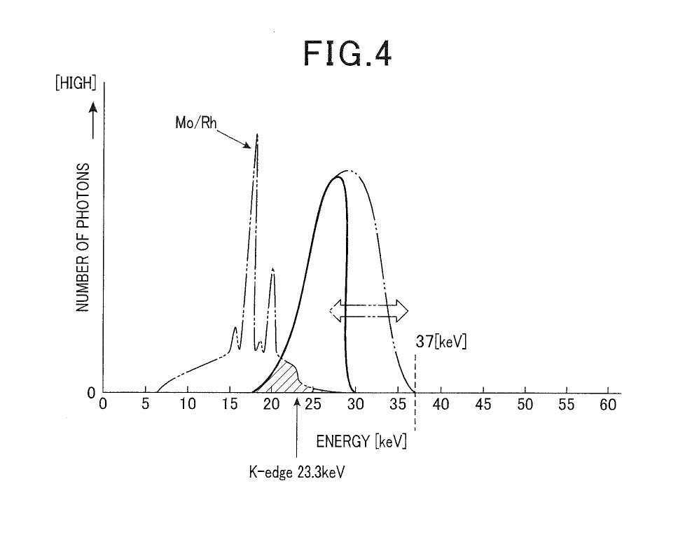

According to the present embodiment, the center band of 18 keV to 30 (37) keV, serving as the X-ray band to be used, may be shifted. The point with regard to creating the desired X-ray spectrum is that the energy band used in mammography in the present invention is sufficiently higher than the energy band (roughly 10 keV to 23 keV) used in conventional mammography. As an indicator thereof, according to the present embodiment, the inventors of the present invention and the like propose the use of an energy band that at least has an average X-ray energy that is higher than that of the energy band used by conventional mammography apparatuses and of which the overlap with the conventional energy range is 20% or less (see the slanted line portion in FIG. 4, described hereafter).

FIG. 4 shows a comparison of the energy spectrum of X-rays emitted from the X-ray generator 21 towards the breast BR of the test subject P and the energy spectrum of X-rays that are mainstream in conventional mammography. In FIG. 4, the energy spectrum for conventional X-ray mammography is that of an example in which molybdenum (Mo) is used as the anode of the X-ray tube and a filter composed of rhodium (Rh) is used as the above-described filter. This energy spectrum is indicated as Mo/Rh.

Upon comparison of the two spectrums shown in FIG. 4, that is, the spectrum according to the present embodiment and the spectrum Mo/Rh of the conventional example, the difference therebetween is clear. The two spectrums according to the present embodiment both have energy bands towards the higher-range side (mainly 18 to 30 (37) keV) than that of the conventional example, and have a continuous distribution with no characteristic X-rays. The energy spectrums indicate a higher X-ray energy than that in the conventional example and are suitable for X-ray mammography.

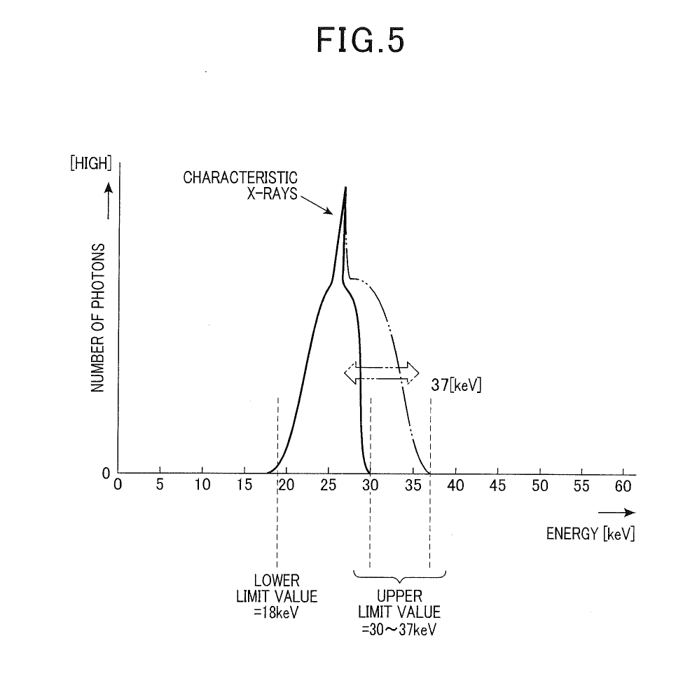

FIG. 5 shows another energy spectrum that is applicable to the present invention. The energy spectrum is that in which a material other than tungsten, such as molybdenum or copper, is used as an anode material 22A of the X-ray tube 22. In this case, a peak formed by characteristic X-rays appears near energy=26 keV. As a result, the number of photons at the energy of the characteristic X-rays can be increased. This enables, for example, optimization of the amount of information required for imaging on the X-ray generation side, when the image contrast by energy near 26 keV is the highest.

In this way, X-rays of which the energy band has been corrected (restricted) are incident on the breast BR of the test subject P from the X-ray generator 21.

Returning to FIG. 1, the compression plates 32A and 32B are configured to sandwich the breast BR of the test subject P between the top surface of the X-ray detection apparatus 31 and compress the breast BR. A reason for this is to enable a more detailed visualization of a legion by the breast BR being imaged in a state in which the breast BR is deformed to the thinnest state possible.

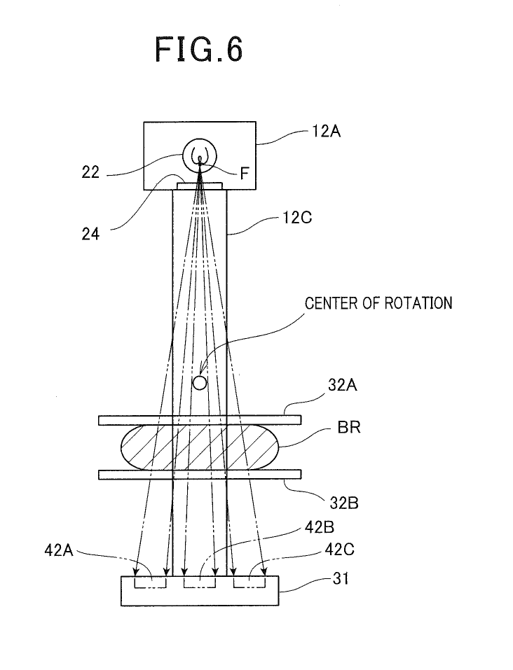

FIG. 6 shows a geometric positional relationship, mainly of the X-ray tube 22, the collimator (slit) 24, the breast BR, and a detector 42 (described hereafter), when the gantry 11 shown in FIG. 1 is viewed from the front direction (the direction of arrow FR).

In addition, the X-ray detection apparatus 31 includes a grid 41, the X-ray detector (referred to, hereafter, as simply a detector) 42, and a bias power supply 43. The grid 41 is used to prevent scattered radiation of X-rays. The detector 42 detects the X-rays. The bias power supply 43 supplies a high-voltage bias voltage to the detector 42.

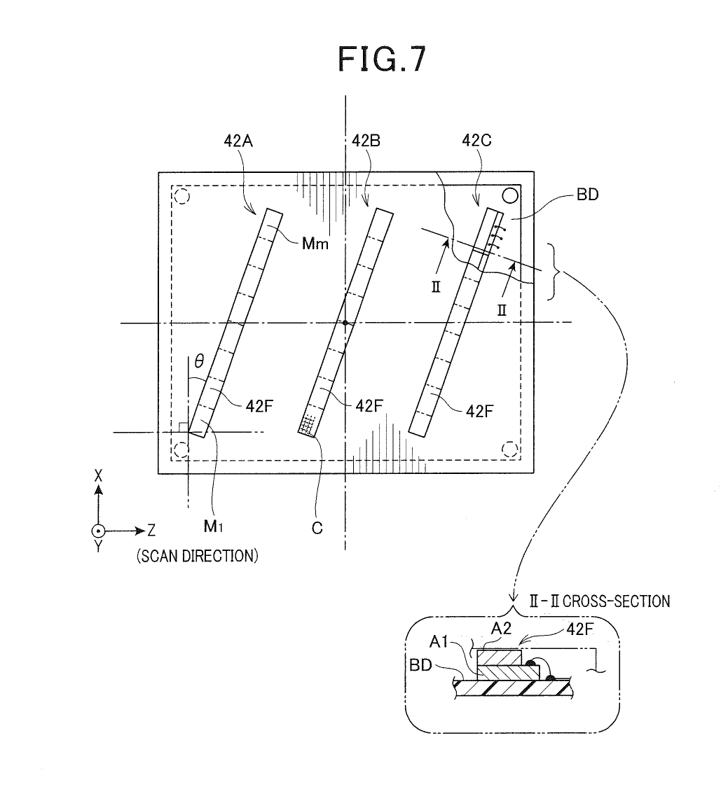

As shown in FIG. 7, the detector 42 has a substrate BD and three detectors 42A to 42C that each have an elongated rectangular shape. The detectors 42A to 42C are mounted on the substrate BD such as to be separated from each other by a predetermined distance and parallel to each other. X-ray image sensors are arrayed in a two-dimensional manner on the detectors 42A to 42C. Each of the three detectors 42A to 42C provide a detection surface 42F. The three detectors 42A to 42C are formed as blocks that are independent of each other and mounted on the substrate BD. As a result of the three detectors 42A to 42C being disposed in a dispersed manner in this way, compared to a detector configuration in which X-ray image sensors are arrayed over the overall area including the spaces between the detectors 42A to 42C, the component cost of the detector can be reduced and incidence of scattered rays can be suppressed.

Of course, a single detector that covers a two-dimensional area of a required size can also be used as required.

Each detector 42A (to 42C) is configured as a direct-conversion-type, photon-counting-type X-ray detector composed of a semiconductor.

Specifically, each detector 42A (to 42C) is configured as an elongated-shaped detector such that a plurality of detection modules M₁ to Mₘ are disposed in a vertical row with a gap of a predetermined width in one direction. Each detector 42A (to 42C) is tilted on the substrate BD by 0° (such as 16.5°) in relation to a direction perpendicular to the scan direction. As shown in FIG. 7, each detection module M₁ (to Mₘ) has collection pixels C (such as 12×80 pixels) that are arrayed in a two-dimensional manner. As a result, the collection pixels C are also disposed such as to be tilted at an angle of 0° in relation to a direction orthogonal to the scan direction, that is, to the scan direction itself. Therefore, even when a gap is present between the detection modules M₁ to Mₘ, the collection pixels C are arrayed over the overall area of the desired imaging range in the direction perpendicular to the scan direction. That is, signals can be collected with certainty even from a section corresponding to the gap.

The collimator 24 is formed such that the X-rays are radiated onto only the respective detection surfaces 42F, positioned at an angle, of the three detectors 42A to 42C.

Each detection module M₁ (to Mₘ) includes an application-specific integrated circuit (ASIC) layer A1 and a detection layer A2. The ASIC layer A1 is mounted on the substrate BD. The detection layer A2 is joined by bonding to the ASIC layer A1.

In each detector 42A (to 42C), for example, ten detection modules M are arranged in a linear manner. Therefore, the collection pixels C (such as 12×80 pixels) are provided for each detector. The size of each collection pixel C is, for example, 200 μm×200 μm. In addition, the size of the X-ray detection surface of each detector 42A (to 42C) is, for example, 4 mm wide×160 mm long).

Therefore, in the detector 42, an N-number of pixels that configure an incidence surface 42F (that is, the detection surface) of each detector 42A (to 42C) individually count photons based on the incident X-rays. The detector 42 then outputs data of the electric quantity reflecting the count value at a high frame rate of, for example, 300 to 3300 fps. The data is also referred to as frame data.

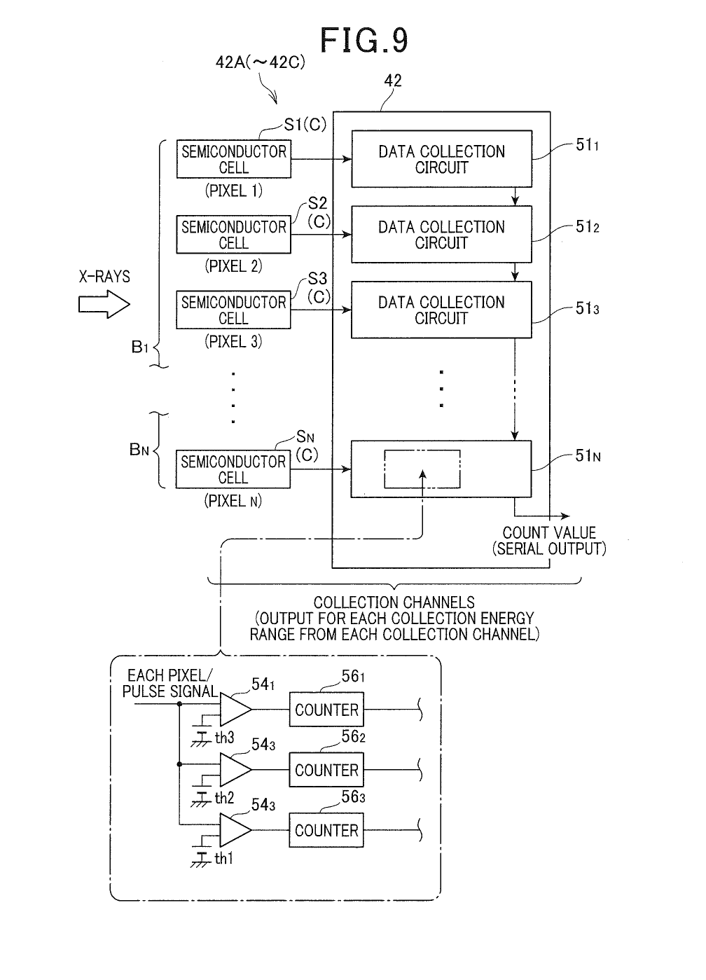

Each of the plurality of collection pixels C is composed of a semiconductor cell (sensor) Sn (n=1 to N), such as a cadmium telluride semiconductor (CdTe semiconductor), cadmium zinc telluride semiconductor (CdZnTe semiconductor), silicon semiconductor (Si semiconductor), or CsI. Each semiconductor cell Sn detects incident X-rays and outputs pulsed electrical signals based on the energy value of the X-rays. That is, the detector 42A (to 42C) is provided with a cell group in which a plurality of semiconductor cells Sn are arrayed in a two dimensional manner. A data collection circuit 51ₙ (n=1 to N) is provided on the output side of each of the semiconductor cells Sn, that is, each of the plurality of collection pixels C (such as an N-number of collection pixels C, from 1 to N) in the two-dimensional array (see FIG. 9).

An X-ray detection material composing each collection pixel C may be an element in which a scintillator that has a fast decay time and uses crystals, such as praseodymium-doped lutetium aluminum garnet (Pr:LuAG) or gadolinium aluminum gallium garnet (Ce:GAGG), is combined with a photoelectric conversion element such as a silicon photomultiplier (SiPM).

The structure of the group of semiconductor cells Sn is also known through JP-A-2000-69369, JP-A-2004-325183, and JP-A-2006-101926.

The above-described size (200 μm×200 μm) of the collection pixel C is set to a value that is small enough to enable detection of X-rays as particles (X-ray photons) and the number of particles. According to the present embodiment, the size enabling detection of X-rays as these particles is defined as “a size enabling the occurrence of a superposition phenomenon (pile-up) between pulsed electrical signals in response to each incidence event when radiation (such as X-ray) particles are continuously incident in plural numbers at the same position or near the position to be essentially disregarded, or enabling the amount thereof to be predicted”. When the superposition phenomenon occurs, a counting loss of X-ray particles (also referred to as a pile-up count loss) occurs in the characteristics of “incidence count versus actual counted number” of X-ray particles. Therefore, the size of the collection pixel C in each detector 42A (to 42C) is set to a size at which counting loss does not occur or can be considered to essentially not have occurred. Alternatively, the size is set to an extent enabling estimation of the amount of count loss. This characteristic of the detector 42A (to 42C) is that the number of X-ray pulses can be accurately counted while accurately performing energy differentiation.

Next, circuits that are electrically connected to the detector 42A (to 42C) will be described with reference to FIG. 9. Each of the plurality of data collection circuits 51ₙ (n=1 to N) has a charge amplifier that receives an analog-quantity electrical signal that is outputted from each semiconductor cell. At stages following the charge amplifier, the data collection circuit 51 includes a waveform rectifying circuit, multiple stages of comparators, multiple stages of counters, multiple stages of digital-to-analog (D/A) convertors, a latch circuit, a serial convertor, and the like. These circuit configurations are known through JP-A-2006-101926.

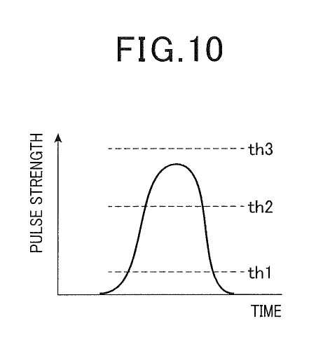

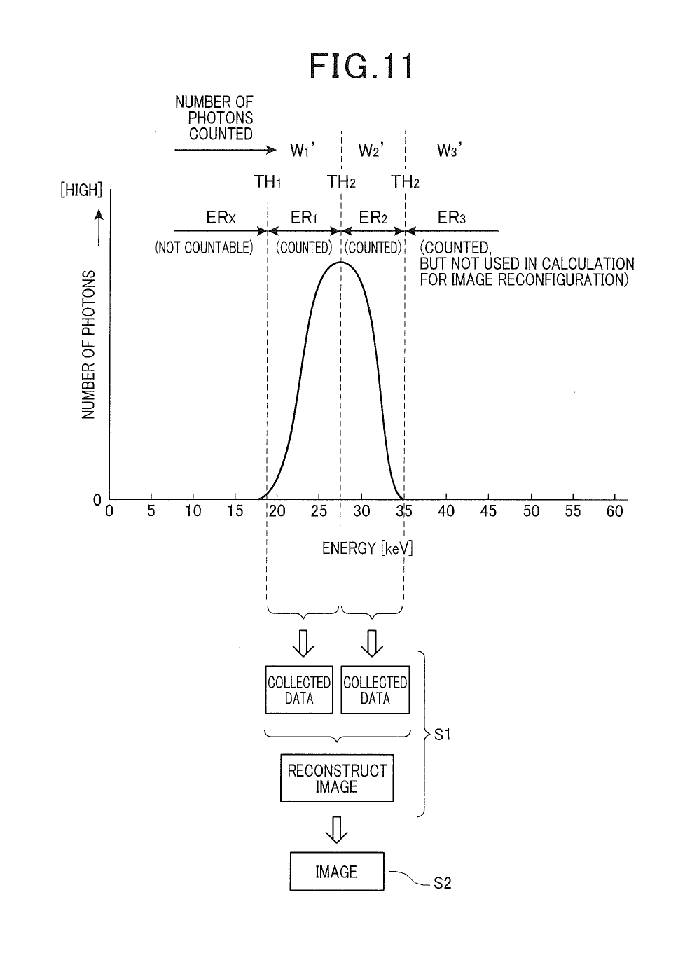

The main sections are as follows. In the data collection circuit (n=1 to N), an output terminal of the waveform rectifying circuit is connected to a comparison input terminal of each of, for example, three stages of comparators 54₁ to 54₃. As shown in FIG. 10, analog-quantity thresholds thᵢ (here, i=1 to 3) having differing values are respectively applied to the respective reference input terminals of the three comparators 54₁ to 54₃. As a result, a single pulse signal can be separately compared with the differing analog-quantity thresholds th₁ to th₃. A reason for this comparison is to determine (differentiate) the range, among energy ranges ER_{EX} and ER₁ to ER₃ (also referred to as BINs; see FIG. 11) that have been divided into three and set in advance, to which the energy value of the incident X-ray particle belongs. A determination is made regarding the value, among the analog-quantity thresholds th₁ to th₃, that the wave peak of the pulse signal (that is, the energy value of the incident X-ray particle) exceeds. As a result, the energy range ER_{EX} and ER₁ to ER₃ to which the differentiation is made differs.

The lowest analog-quantity threshold th₁ is ordinarily set as a threshold for preventing detection of disturbances, noise attributed to the semiconductor cell Sn or circuits such as the charge amplifier, or low-energy radiation that is not necessary for imaging. According to the present embodiment, this threshold th₁ is set to a value corresponding to the lower limit value=18 keV of the energy band required for imaging. Therefore, the band ER_{EX} of which the energy is lower than the lowest analog-quantity threshold 18 keV is considered a “not-countable (uncounted) range” due to a large amount of information being affected by noise and disturbances. Meanwhile, the number of photons in the highest energy range ER₃ is counted. However, the count is handed as a value that is not used for image reconfiguration. Here, the highest analog-quantity threshold th₃ is set to a desired value within range of the tube voltage=30 to 37 kV to enable determination of the superposition phenomenon (pile-up) caused by cosmic rays. In the example in FIG. 11, the analog-quantity threshold th₃ is set to 35 kV.

Therefore, according to the present embodiment, as the number of X-ray photons, the X-ray photons having energy belonging to the two energy ranges in the middle, that is, first and second energy ranges ER₁ and ER₂ are counted. Specifically, counters 56₁ to 56₃ that are disposed in each data collection circuit 51ᵣ, respectively count the number of photons having energy that belongs to the first (to third) energy range ER₁ (to ER₃), of which the counter is to perform counting, or energy exceeding the energy range. Therefore, when the respective numbers of X-ray photons having energy belonging to the first to third energy ranges ER₁ to ER₃, that is, the number of X-ray photons determined for each energy range is W₁, W₂, and W₃, the relationship with the count values W₁′, W₂′, and W₃′ of the first to third counters 56₁ to 56₃ is

W₁=W₁′−W₂′ and

W₂=W₂′−W₃′.

Here, W₃=W₃′ is meaningless information (that is, the energy range of the X-ray photons is unidentifiable) attributed only to a superposition phenomenon occurring with a small amount of cosmic rays. Therefore, although the value is known, the value is not used for image generation.

Here, the count values W₁ to W₂ that are truly desired are determined by a subtraction process based on the above-described expressions by a data processor, described hereafter. Ideally, W₃=W₃′=0.

In this way, according to the present embodiment, the numbers of X-ray photons W₁ to W₂ respectively belonging to the first to second energy ranges ER₁ to ER₂ are determined by calculation (subtraction) from the actual count values W₁′ to W₃′. As a result, the circuit configuration mounted in the data collection circuit 51ₙ can be simplified.

Therefore, the meaning of “collection” of the number of X-ray photons for each energy range in the present application includes both “determination by calculation” from the actual count value, as described above, and directly “counting” the number of X-ray photons for each energy range, such as in a variation example described hereafter.

The above-described counters 56₁ to 56₃ are provided with signals for startup and stop from a controller, described hereafter, of the console 4. Counting over a certain amount of time is managed externally through use of a reset circuit provided in the counter itself.

In addition, the number of thresholds, that is, the number of comparators is not necessarily limited to three. The number of thresholds may be two including the analog-quantity threshold th₁, described above, or may be any quantity that is three or more. The number of thresholds is dependent on the number of energy ranges for which the number of X-ray photons is counted, also referred to as BINs. When the number of energy ranges is one, there are two thresholds, th₁ and th₂. When this configuration is implemented in the present example, th₁=a reference voltage value corresponding to 18 keV and thₑ=a reference voltage value corresponding to 30 (to 37) keV. In addition, when the number of energy ranges to be subjected to counting is three, there are four thresholds, th₁, th₂, th₃, and th₄. When this configuration is implemented in the present example, th₁=a reference voltage value corresponding to 18 keV and th₄=a reference voltage value corresponding to 30 (to 37) keV. thₑ and th₃=appropriate reference voltage values corresponding to appropriate energy amounts selected from 18 to 30 (to 37) keV, respectively. That is, in the example in FIG. 11, the energy range 18 to 30 keV is differentiated into three energy ranges. The X-ray photons are counted for each range. When the number of energy ranges to be subjected to counting is four, in a similar manner, in the example in FIG. 11, the energy range 18 to 30 keV is differentiated into four energy ranges. The X-ray photons are counted for each range.

Returning to the present embodiment, specifically, the above-described analog-quantity thresholds th₁ to th₄ are provided as digital values from the console 4, as values that have been calibrated for each collection pixel C, or in other words, for each collection channel.

In this way, during a certain collection time that is reset at a certain cycle, the number of particles of the X-rays incident on each detector 42A (to 42C) is counted by the three counters 56₁ to 56₃, for each collection pixel C and for each energy range. The count values of the numbers of X-ray particles are respectively outputted in parallel as digital-quantity count data W₁′, W₂′, and W₃′ from the first to third counters 56₁ to 56₃. Thereafter, the count values are converted to serial format by a serial converter (not shown). The serial converter is serially connected to the serial converters of all other collection channels. Therefore, all pieces of digital-quantity count data are outputted serially from the serial converter of the last channel and sent to the console 4.

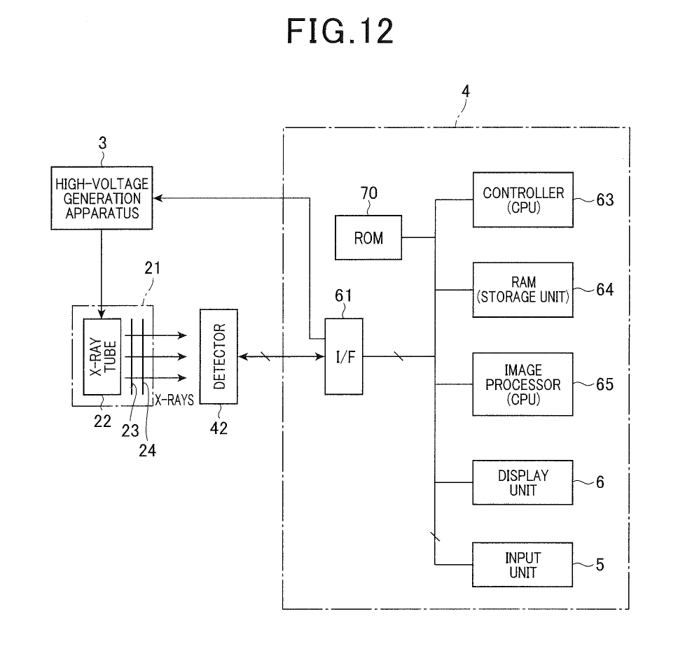

As shown in FIG. 12, the console 4 includes an interface (I/F) 61 that handles input and output of signals. The console 4 also includes a controller (central processing unit (CPU)) 63, a random access memory (RAM) (storage unit) 64, an image processor 65, and a read-only memory (ROM) 70 that are communicably connected to the interface 61 by a bus 62. In addition, the interface 61 is connected to the input unit 5 and the display unit 6, and is able to communicate with the controller 63.

The controller 63 controls the driving of the gantry 11 based on a program provided in the ROM 70 in advance. The control includes a send-out command of a command value to the high-voltage generation apparatus 3. The RAM 64 temporarily stores frame data sent from the gantry 11 via the interface 61.

The image processor 65 performs various processes based on a program provided in the ROM 70 in advance, under the control of the controller 63.

The processes include a process in which a publicly known CT reconfiguration method is performed or a process in which a tomosynthesis method that is referred to as shift-and-add is performed. As a result of these processes, a tomographic image of a desired cross-section of the breast BR of the test subject P is generated through use of the frame data based on the count value of the number of X-ray photons collected for each energy range that is outputted from each detector 42A (to 42C).

The display unit 6 displays the image generated by the image processor 65. In addition, the display unit 6 also handles display of information indicating the operating state of the gantry 11 and operator-operation information provided via the input unit 5. The input unit 5 is used by the operator to provide the system with information required for imaging.

The controller 63 and the image processor 65 include CPUs (central processing unit) that operate based on provided programs. The programs are stored in the ROM 70 in advance.

In the X-ray mammography apparatus 1 configured as described above, the arm portion 12 of the gantry 11 is rotated or revolved around the breast BR of the test subject P under the control of the controller 63. During rotation, the X-rays from the X-ray generator 21 are radiated towards the breast BR to be imaged.

As described above, the energy spectrum of the X-rays are corrected by the aluminum filter 23. That is, the spectrum is corrected as shown in FIG. 3. Based on the corrected spectrum, the X-rays have broad energy over a band of about 18 to 30 (or, to 35) keV. That is, in a band lower than about 18 keV, energy is substantially cut by the aluminum filter 23. X-rays having energy primarily over the band of about 18 to 30 (or, to 37) keV passes through the breast BR that is soft tissue.

Some of the photons of the X-rays are absorbed by the tissue of the breast BR. However, the remaining photons, the amount of which is greater than in the past, pass through the breast BR and are detected by the detectors 42A to 42C. As a result, data in which X-rays are directly converted to digital electric quantity, that is, the above-described frame data, is outputted from the detectors 42A to 42C. The frame data is data reflecting the cumulative value of the number of X-ray photons for each energy range ER of each collection pixel C.

The frame data is collected for each frame at a certain frame rate while the arm portion 12 is rotating around a center of rotation (see FIG. 6), or revolving or moving within a certain area. The frame data is successively sent to the console 4 and stored in the RAM 64.

Then, when imaging, that is, data collection is completed, the image processor 65 reads out the frame data stored in the RAM 64 based on a command from the operator from the input unit 5. The image processor 65 uses the frame data to reconstruct an image, such as an X-ray transmission image of a certain cross-section of the breast BR, based on the tomosynthesis method. Frame data for two energy ranges ER₁ and ER₂ are obtained from each collection pixel C.

Therefore, in reconfiguration of the image, for example, the image processor 65 gives a little or zero weight to the frame data of the high energy range ER₂, and gives greater weight to the frame data of the low energy range ER₁. The image processor 65 then adds the frame data together for each collection pixel C. As a result, collected data is generated for each collection pixel C. As a result, data accompanying X-ray scanning that has been collected from all collection pixels C are gathered. Therefore, the collected data is processed by a suitable reconfiguration method and an image of the breast BR is reconstructed (step S1 in FIG. 11). The panoramic image is, for example, displayed in the display unit 36 (step S2 in FIG. 11). Of course, the image may be reconstructed without weighting being performed.

Here, electrical noise in the detection circuit of the X-rays, described above, will be described. From the perspective of eliminating the effect of electrical noise, apparatuses that are mounted with photon-counting-type detectors have recently been commercialized. In the photon-counting detection technique, X-rays are considered particles. Rectification to pulse signals that allow the energy of the particles to be viewed as pulse height is performed. As a result of an energy threshold being provided, only pulses exceeding the threshold are counted. A system in which counting is performed independently by pixels that are about 200 μm×200 μm, and that is capable of differentiating between a plurality of energy thresholds is commercialized. In this technique, the threshold is set at energy that is at least higher than electrical noise. Therefore, a significant characteristic is that electrical noise is not present.

In addition, collection that is divided into a plurality of energy ranges is possible. Therefore, because the X-ray absorption coefficients of substances differ depending on the X-ray energy, contrast tends to be more easily obtained through high energy for substances having a high X-ray absorption coefficient. Contrast tends to be more easily obtained through low energy for substances having a low X-ray absorption coefficient. Therefore, as a result of weighted addition being performed accordingly on the images for each BIN, processing methods enabling both tumors and calcifications to be optimally displayed, or either of the tumors and calcifications to be obtained at maximum contrast can be performed.

As described above, in the X-ray mammography apparatus according to the present embodiment, problems faced by conventional integrating-type X-ray detectors can be improved. That is, for example, the SN ratio is poor as a result of electrical noise. In addition, contrast is required to be ensured through reduction of X-ray energy, regardless of high X-ray exposure dose to the patient, because the ability to differentiate contrast at high dose regions and low dose regions is insufficient due to the narrow dynamic length of the circuit.

In addition, regarding an issue in which a pixel size of 100 μm or less is relatively difficult to achieve due to the large amount of circuit implementation in the photon-counting-type detector, a small-focal-point X-ray tube is used, and magnification effect and phase contrast effect are actualized, thereby solving the problem of resolution required for visualizing calcification. Optimization of imaging of calcification that has a relatively significantly high X-ray absorption coefficient and mass that has a low X-ray absorption coefficient, which is a characteristic feature of X-ray mammography, can be achieved.

In addition, in the X-ray detection method, the photon-counting-type detector that is capable performing output with the energy band divided into at least two bands is used. Image resolution has a resolution that is twice the subject or test subject to be determined, or less. The X-ray generator has, for example, a filter that is disposed in an X-ray tube that has an anode. The filter suppresses transmission of X-ray particles having energy in bands higher than the above-described energy spectrum. The X-ray tube focal point size is 0.056 mm or less. The subject or test subject is separated from the X-ray tube focal point position by 0.5 m or more, and the distance from the subject or test subject to the detector is set to 0.5 m or more. The phase contrast effect is targeted, contrast emphasis is achieved, and at the same time, resolution is ensured through the magnification effect. Refer to “Konica Minolta Phase Contrast Technology: http://www.konicaminolta.jp/healthcare/technique/contrast.html”, for example, regarding phase contrast.

When images are obtained in which division into a plurality of energy BINs has been performed in the photon-counting-type detector, the energy bands at which optimal contrast can be obtained differs between mass and calcification. Therefore, as a result of weighted addition being performed on the obtained images for each energy band, an optimal image can be obtained. In addition, in the energy band over the energy range from 18 keV to an upper limit of 30 keV to 35 keV, contrast-to-noise ratio (CNR) for optimizing visualization of tumors can be optimized. From this perspective, a technique for optimizing characteristic X-rays is also possible.

### Second Embodiment

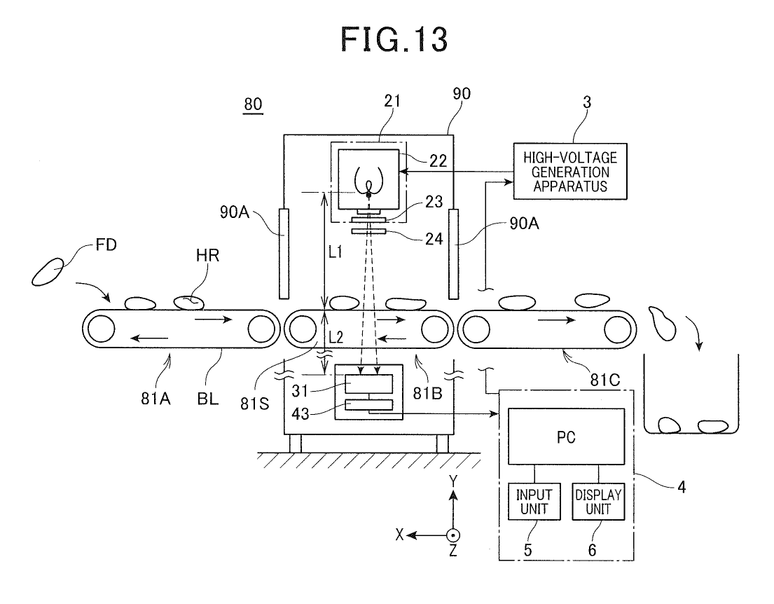

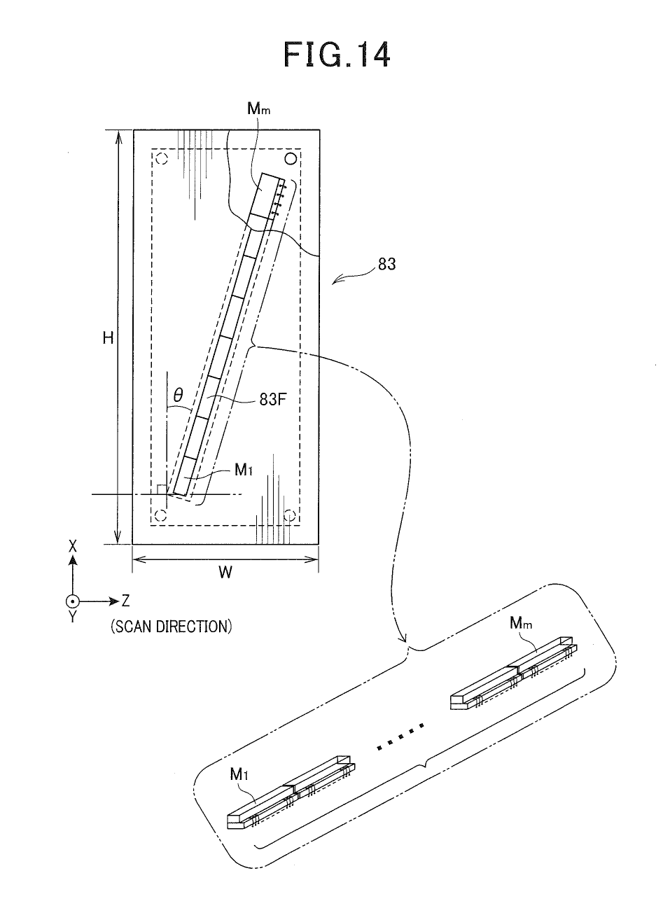

An example of an X-ray foreign matter detection apparatus as the low-energy X-ray image forming apparatus of the present invention will be described with reference to FIG. 13 to FIG. 14.

According to the embodiment, constituent elements that are the same as or equal to those according to the above-described first embodiment are given the same reference numbers. Descriptions thereof are omitted or simplified.

As shown in FIG. 13, the X-ray foreign matter detection apparatus 80 is an apparatus that uses X-rays to detect human hair HR as foreign matter that may be present inside or around a food product FD (a substance corresponding to human soft tissue from the perspective of contrast-to-noise ratio (CNR)) that is placed on and conveyed by conveyor belts 81A, 81B, and 81C. Therefore, the X-ray foreign matter detection apparatus 80 is provided on the intermediate belt conveyor 81B. The X-ray foreign matter detection apparatus 80 periodically forms an X-ray image, every certain amount of time, without stopping or touching the food product FD that is being conveyed. The X-ray foreign matter detection apparatus 80 detects hair from the image and performs an appropriate process, such as issuing a notification.

The foreign matter detection apparatus 80 has a box-shaped casing 90. Inside the casing 90, the X-ray generator 21 is provided such as to be oriented downward. The collimator 24 is provided on the emission side of the X-ray generator 21. Flexible X-ray shields 90A are provided at the food product entrance and the food product exit of the casing 90.

In addition, an X-ray detector 83 that receives transmitted X-rays is provided under the belt conveyor 81B. In the example shown in FIG. 13, L1=L2 is set.

The detector 83 may be positioned in a space 81S between band-shaped belts BL that move in opposite directions above and below in the height direction (Y-axis direction) of the belt conveyor 81B. The belt BL is composed of a material having radiolucency.

As shown in FIG. 14, the detector 83 is configured such that 29 detection modules M, described above, are arranged in a vertical row in one direction. The detector 83 is arranged such as to be tilted on the substrate BD by θ° (such as 16.5°) in relation to the scan direction, that is, the direction in which the food product FD is conveyed. As a result, for example, the detector 83 has a size with a vertical H=460 mm and a horizontal width W=145 mm.

In comparison with the detectors 42A to 42C according to the first embodiment, described above, the detector 83 according to the second embodiment is the same as those according to the first embodiment, aside from the quantity thereof being one and the number of modules arranged in the vertical row being larger, or in other words, being longer.

The console 4 performs, for example, the shift-and-add process in time with the movement speed of the belt conveyor 81B on the frame data detected at a high-speed frame rate by the detector 83. As a result, for example, a tomographic image is formed at a certain cycle along a virtual plane assumed to be at a position at the same height as a detection surface 83F of the detector 83 or a virtual plane assumed to be at a position at a desired height. The food product FD is captured in the image. If foreign matter such as hair is present, the foreign matter is also captured with the food product in an overlapping state. The console 4 recognizes the foreign matter by a known image recognition method. The console 4 then performs a process, such as issuing a notification to the operator or issuing an instruction to remove the relevant food product FD from the line.

Other configurations are the same as or equal to those according to the first embodiment. Therefore, working effects equivalent to those described above are achieved.

Therefore, in the X-ray foreign matter detection apparatus 80, in addition to the working effects equivalent to those described above, the presence of foreign matter that is difficult to image by conventional X-ray imaging, such as hair and foreign matter that is thin and fine, can be detected through image formation at a high image resolution. In addition, the size of the apparatus can be reduced through shortening of the time required for foreign matter detection and reduction in tube current. In addition, manufacturing cost of the apparatus can also be reduced.

### REFERENCE SIGNS LIST

1 X-ray mammography apparatus (low-energy X-ray image formation apparatus)
3 high-voltage generation apparatus
4 console (image formation means)
21 X-ray generator
22 X-ray tube
22A anode
23 aluminum filter (filter)
31 X-ray detection apparatus
42, 42A to 42C detector
63 controller
64 RAM (storage unit)
65 image processor (CPU)
70 ROM
M₁ (to Mₘ) detection module
Sn semiconductor cell
C collection pixel

1. A low-energy X-ray image formation apparatus, comprising:
an X-ray generator generating X-rays having an energy spectrum showing an energy range continuously ranging from a lower limit energy value to an upper limit energy value, the energy range being higher in energy from an effective energy of an energy range ranging 10 to 23 keV, the lower limit energy value being 18 keV, the upper limit energy value being within a range of 30 to 37 keV;
a detector detecting the X-rays generated by the X-ray generator and transmitted through a soft tissue of an subject being imaged or a tissue of a substance, the tissue of the substance corresponding in a contrast-to-noise ratio (CNR) to the soft tissue of the object;
image forming means for forming an image of the soft tissue of the object or an image of the substance, based on a detection signal outputted from the detector,
wherein the soft tissue and the substance are defined as a soft tissue and a substance presenting the contrast-to-noise ratio (CNR) of 3.8 or more when the X-rays are radiated to the soft tissue and the substance in a condition where an X-ray tube voltage is set at 20 kV.

2. The low-energy X-ray image formation apparatus of claim 1, wherein
the X-ray generator comprises
an X-ray tube having an anode generating the X-rays; and
a filter arranged on an X-ray radiation side in the X-ray tube and arranged at a position through which the X-rays from the anode pass, and
the filer comprises a filter suppressing particles of the X-rays from passing therethrough, the particles of the X-rays having energy values lower than the energy spectrum.

3. The low-energy X-ray image formation apparatus of claim 2, wherein
the X-ray generator is configured to generate the X-rays having an energy spectrum ranging from the lower limit energy value of 18 keV and having no characteristic X-rays in the energy range ranging from the lower limit energy value to an upper limit energy value falling into a range of 30 to 37 keV.

4. The low-energy X-ray image formation apparatus of claim 2, wherein
the X-ray generator is configured to generate, as the X-rays, X-rays having an energy spectrum having a peak of characteristic X-rays in the energy range.

5. The low-energy X-ray image formation apparatus of claim 4, wherein
the X-ray generator is provided with the anode whose material provides the energy spectrum with the energy range ranging from the lower limit value of 18 keV to the upper limit value of 30-37 keV and giving the characteristic X-rays in the energy range.

6. The low-energy X-ray image formation apparatus of claim 1, wherein
the X-ray tube has a focal point size of 0.056 mm or less,
a distance between the focal point of the X-ray tube and the soft tissue of the object or the substance is set at 0.5 m or more, and
a distance between the soft tissue of the object or the substance and the detector is set at 0.5 m or more, a phase contrast effect being given to X-ray image formation.

7. The low-energy X-ray image formation apparatus of claim 1, wherein
the detector is an X-ray detector, every one of pixels of the detector, responding to incidence of particles to output pulsed electrical signals according to values of energy of the particles, the X-rays being regarded as being composed of the particles, and
the X-ray detector is provided with a signal processing circuit outputting the detection signal depending on an amount of the particles of the X-rays, every the one of the pixels and every one of one or more energy ranges provided by removing both an uppermost energy range and a lowermost energy range from three or more energy ranges, the three or more energy ranges being provided by dividing the energy spectrum into three or more energy ranges.

8. The low-energy X-ray image formation apparatus of claim 7, wherein
the X-ray detector is provided with a signal processing circuit outputting the detection signal depending on an amount of the particles of the X-rays, every the one of the pixels in one energy range provided by removing both an uppermost energy range and a lowermost energy range from three or more energy ranges, the three or more energy ranges being provided by dividing the energy spectrum into three or more energy ranges.

9. The low-energy X-ray image formation apparatus of claim 1, wherein
the detector is an elongated-shaped detector in which a plurality of modules are arranged in line with a gap provided between adjacent modules among the modules, two-dimensionally arrayed pixels being arranged in each of the modules, the pixels being composed of elements to detect the X-rays.

10. The low-energy X-ray image formation apparatus of claim 9, wherein
the elongated-shaped detector is a plurality of elongated-shaped detectors which are arranged discretely parallel to each other.

11. The low-energy X-ray image formation apparatus of claim 9 or 10, wherein
the elongated-shaped detector is arranged to obliquely to a direction, the direction being orthogonal to a direction in which the X-rays are moved relatively to the soft tissue of the object or the substance, a row of the pixels in a longitudinal direction being oblique to the orthogonal direction.

12. The low-energy X-ray image formation apparatus of claim 1, wherein
the soft tissue of the object is a breast of a human body, and
the low-energy X-ray image formation apparatus is provided as an X-ray mammography apparatus.

13. The low-energy X-ray image formation apparatus of claim 1, wherein
the low-energy X-ray image formation apparatus is provided as an X-ray foreign matter detection apparatus detecting a foreign matter which may exist within or around the substance.

14. A method of forming an image based on low-energy X-rays, comprising steps of:
generating X-rays having an energy spectrum showing an energy range continuously ranging from a lower limit energy value to an upper limit energy value, the energy range being higher in energy from an effective energy of an energy range ranging 10 to 23 keV, the lower limit energy value being 18 keV, the upper limit energy value being within a range of 30 to 37 keV;
detecting the X-rays generated by an X-ray generator and transmitted through a soft tissue of an subject being imaged or a tissue of a substance, the tissue of the substance corresponding in a contrast-to-noise ratio (CNR) to the soft tissue of the object; and
forming an image of the soft tissue of the object or an image of the substance, based on a detection signal outputted from the detector,
wherein the soft tissue and the substance are defined as a soft tissue and a substance presenting the contrast-to-noise ratio (CNR) of 3.8 or more when the X-rays are radiated to the soft tissue and the substance in a condition where an X-ray tube voltage is set at 20 kV.

15. The low-energy X-ray image formation apparatus of claim 2, wherein
the X-ray tube has a focal point size of 0.056 mm or less,
a distance between the focal point of the X-ray tube and the soft tissue of the object or the substance is set at 0.5 m or more, and
a distance between the soft tissue of the object or the substance and the detector is set at 0.5 m or more, a phase contrast effect being given to X-ray image formation.

16. The low-energy X-ray image formation apparatus of claim 15, wherein
the detector is an X-ray detector, every one of pixels of the detector, responding to incidence of particles to output pulsed electrical signals according to values of energy of the particles, the X-rays being regarded as being composed of the particles, and
the X-ray detector is provided with a signal processing circuit outputting the detection signal depending on an amount of the particles of the X-rays, every the one of the pixels and every one of one or more energy ranges provided by removing both an uppermost energy range and a lowermost energy range from three or more energy ranges, the three or more energy ranges being provided by dividing the energy spectrum into three or more energy ranges.

17. The low-energy X-ray image formation apparatus of claim 16, wherein
the X-ray detector is provided with a signal processing circuit outputting the detection signal depending on an amount of the particles of the X-rays, every the one of the pixels in one energy range provided by removing both an uppermost energy range and a lowermost energy range from three or more energy ranges, the three or more energy ranges being provided by dividing the energy spectrum into three or more energy ranges.

18. The low-energy X-ray image formation apparatus of claim 15, wherein
the detector is an elongated-shaped detector in which a plurality of modules are arranged in line with a gap provided between adjacent modules among the modules, two-dimensionally arrayed pixels being arranged in e of the modules, the pixels being composed of elements to detect the X-rays.

19. The low-energy X-ray image formation apparatus of claim 18, wherein
the elongated-shaped detector is a plurality of elongated-shaped detectors which are arranged discretely parallel to each other.
